(19) United States
(12) Patent Application Publication
(10) Pub. No.: US 2026/0022333 A1
(43) Pub. Date: Jan. 22, 2026

(54) IMPARTING UNIQUE CELL-SCAFFOLD INTERACTIONS TO CREATE SELF-ORGANIZING TISSUES

(71) Applicant: William Marsh Rice University, Houston, TX (US)

(72) Inventors: Kevin J. McHUGH, Houston, TX (US); Marina H. YU, Houston, TX (US)

(73) Assignee: William Marsh Rice University, Houston, TX (US)

(21) Appl. No.: 19/272,210

(22) Filed: Jul. 17, 2025

Related U.S. Application Data
(60) Provisional application No. 63/672,376, filed on Jul. 17, 2024.

(51) Int. Cl. C12N 5/00

(52) U.S. Cl. CPC C12N 5/0068; C12N 2513/00; C12N 2533/50; C12N 2533/74; C12N 2535/10

(57) ABSTRACT

The present disclosure is directed to scaffold materials patterned with cell adhesion targets that permit the attachment and growth of predetermined cell types onto the scaffold. By selecting specific ligands that are patterned on the scaffold, particular cell types, including those engineered for this specific purpose, will attach and proliferate to create designed tissues and organs.

### STATEMENT REGARDING FEDERALLY SPONSORED RESEARCH AND DEVELOPMENT

This invention was made with government support under Grant No. 1842494 awarded by the National Science Foundation. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Jul. 16, 2025, is named RICEP0157US.xml and is 5,461 bytes in size.

### PRIORITY CLAIM

This application claims benefit of priority to U.S. Provisional Application Ser. No. 63/672,376, filed Jul. 17, 2024, the entire contents of which are hereby incorporated by reference.

### BACKGROUND

### 1. Field of the Disclosure

The present disclosure relates generally to the fields cell biology, cell engineering and artificial tissues/organs. More particularly, the disclosure relates to compositions comprising patterned scaffolds that contain cell adhesion targets for attachment and growth of specific cells types into artificial tissues and organs.

### 2. Background

The field of tissue engineering has rapidly grown in an attempt to meet the clinical demand for healthy tissues. There is a limited supply of tissues and organs and only a small fraction are suitable for transplantation. Recent advances have enabled the production of several synthetic tissue types; however, these tissues are not as complex as the native tissue they seek to mimic. Cell adhesion motifs, typically found in extracellular matrix proteins, are increasingly used in scaffolds to improve cell adhesion and to better recapitulate the native microenvironment. The most widely used cell adhesion peptide is RGD, which binds to many cell integrin heterodimers, meaning the presence of RGD enables almost ubiquitous adhesion across all cell types. This is beneficial if the goal is to have multiple cell types adhere to a scaffold without imparting organization, but is detrimental if maintaining a particular tissue architecture is critical to the tissue's function. Thus, improved methodology for preparing engineered tissues is needed.

### SUMMARY

Thus, in accordance with the present disclosure, there is provided a composition comprising a scaffold material patterned with one or more target molecules that bind(s) a cell adhesion protein. The cell adhesion protein may be an integrin, a selectin or a cadherin, such as wherein the integrin is α₄β₁ integrin, α₂β₁ integrin, α₃β₁ integrin, α₇β₁ integrin, and/or α₉β₁ integrin. The target molecule(s) may be a fibronectin peptide, such as LDV. The target molecule(s) may be attached to the scaffold by direct attachment or through a linker, such as a degradable linker. The scaffold material may be polymer, such as hydrogel, such an alginate hydrogel. A plurality of cells may be patterned on said scaffold through binding to said target molecule, such as wherein (a) said plurality of cells have an identical genotype/phenotype, or (b) wherein said plurality of cells have more than one genotype/phenotype. The plurality of cells may be engineered to express one or more cell adhesion proteins, such as α₄β₁ integrin, α₂β₁ integrin, α₃β₁ integrin, α₇β₁ integrin, and/or α₉β₁ integrin, or to not express an integrin, such as α₄β₁ integrin, α₂β₁ integrin, α₃β₁ integrin, α₇β₁ integrin, a selectin or a cadherin, such as E-cadherin or P-selectin. The plurality of cells may be engineered to express an integrin, a selectin and a cadherin and to lack expression of an integrin, a selectin or a cadherin. The plurality of cells may have an identical genotype/phenotype, or more than one genotype/phenotype.

In another embodiment, there is provided a method of preparing a composition according to any one of claims 1-6, comprising (a) providing a scaffold; and (b) patterning one or more targets molecules that bind a cell adhesion protein on a surface of the scaffold. The method may further comprise contacting the patterned scaffold with a plurality of cells, wherein (a) said plurality of cells have an identical genotype/phenotype, or (b) wherein said plurality of cells more than one genotype/phenotype. The plurality of cells may be engineered to express and/or not express one or more cell adhesion peptides (an integrin, such as α₄β₁ integrin, α₂β₁ integrin, α₃β₁ integrin, α₇β₁ integrin, and/or α₉β₁ integrin, a selectin, a cadherin, e.g., E-cadherin or P-selectin), proteins, or small molecules present on the scaffold. The plurality of cells may be engineered to express one or more cell adhesion peptides (an integrin, such as α₄β₁ integrin, α₂β₁ integrin, α₃β₁ integrin, α₇β₁ integrin, and/or α₉β₁ integrin, a selectin, a cadherin, e.g., E-cadherin or P-selectin), proteins, or small molecules present on the scaffold. The plurality of cells may be engineered to not express one or more cell adhesion peptides (an integrin, such as α₄β₁ integrin, α₂β₁ integrin, α₃β₁ integrin, α₇β₁ integrin, and/or α₉β₁ integrin, a selectin, a cadherin, e.g., E-cadherin or P-selectin), proteins, or small molecules present on the scaffold.

Further provided is an artificial tissue or organ produced by culturing a cell containing composition as described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Peptide sequences and integrin heterodimer pairs (SEQ ID NOs: 1-5).

FIG. 2. Flow cytometry data of HEK 293 wild-type (WT) and α₄-knock out (KO) cells in the presence of AlexaFluor-647 conjugated α₄ or α₄β₁ antibodies. Shows HEK WT cells express α₄ and α₄β₁, whereas HEK KO cells express no α₄ β₁ or α₄β₁ (signal overlaps with no stain control).

FIG. 3. Cell adhesion assay. WT cells are almost all spread on the LDV peptide surface and virtually no cells are spread on the LDV scramble peptide surface (control). KO cells express no α₄ so have no ability to bind to the LDV peptide, and the inventors see no cell spread above control (scramble peptide).

FIG. 4. Patterned surface. The left half is patterned with the LDV peptide, while the right half is patterned with the RGD peptide. Cells that express α₄β₁ will be able to bind to the entire surface, while cells that do not express α₄β₁ will only be able to bind to the RGD patterned section. HEK WT can adhere to both sides while HEK KO can only adhere to RGD side.

FIG. 5. Micrographs of patterned surfaces. (Left) No pattern because HEK WT cells can bind and spread on LDV and RGD. (Right) HEK KO cells cannot bind to the LDV side so are only on the RGD side.

FIG. 6. Flow cytometry data of HCT wild-type and HCT α₄-KO cells in the presence of AlexaFluor-647 conjugated α₄ or α₄β₁ antibodies. Shows HCT WT cells do not express α₄ or α₄β₁, whereas HCT KI cells express α₄ and α₄β₁ (shift to the right from WT signal overlapping with no stain control).

FIG. 7. Cell adhesion assay. WT cells have no α₄ so have no ability to bind to the LDV peptide, and inventors see no cell spread above control (scramble peptide). KI cells now have the ability to bind to LDV and the inventors see significantly more spread cells on LDV than scramble. This % can be increased by increasing α₄ and α₄β₁ expression.

FIG. 8. Western blot. Shows α₄ expression in HEK WT and HCT KI cells but no expression in HEK KO and HCT WT cells. Antibody for α₄ and β-actin were used:

α₄—150 kD β-actin—42 kD, fragment at 30 kD

Band intensity of α₄ was normalized to the housekeeping gene band intensity to show how much α₄ was being expressed compared to β-actin (bar graph).

FIG. 9. RT-pPCR. qPCR confirmed α₄ insertion in HCT (HCT KI)—Cq went from non-existent (no expression of α₄ in HCT WT) to ˜24. Unable to confirm KO using qPCR because one base pair mismatch still allows for amplification, flow cytometry and western blot were used to confirm HEK KO.

### DESCRIPTION OF ILLUSTRATIVE EMBODIMENTS

As discussed above, while the field of tissue engineering has made significant advances, there remain limitations that hamper development of tissues for a wide variety of purposes. As a result, there is a worldwide shortage of transplantable tissues and organs. Thus, there is a great need for increased supplies of tissues and organs. Scientists have successfully created a few tissue types in the lab that are meant to be used in place of transplanting tissue directly from another human being. These tissues have not been able to precisely mimic native tissue with its complex structures and functions.

To overcome the limitations of classical scaffold patterning, which largely offers only binary cell adhesion (e.g., broadly cell adhesive regions with RGD), the inventors sought to engineer cell-type specific adhesion that enables only the correct cell type to adhere to each desired region of the scaffold. Because cells express many different integrin heterodimers, they engineered cells using CRISPR-Cas9 to knock in or knock out integrins, as necessary. They used a sequence from fibronectin, containing the tripeptide LDV, that binds only to integrin heterodimer α₄β₁ to establish a workflow for editing cell lines to bind or not bind specific peptides. Essentially, printing cells in a broadly cell-adhesive matrix does not prevent the cells from migrating out immediately after. The patterning is great initially but then cells infiltrate the hydrogel in all directions shortly after, losing the structure that was initially imparted. Thus, the goal here is to keep cells (biologically) confined within their region, which is very important for tissues comprised of multiple cell types.

These and other aspects of the disclosure are described in detail below.

### I. Integrins

Integrins are transmembrane receptors that help cell-cell and cell-extracellular matrix (ECM) adhesion. Upon ligand binding, integrins activate signal transduction pathways that mediate cellular signals such as regulation of the cell cycle, organization of the intracellular cytoskeleton, and movement of new receptors to the cell membrane. The presence of integrins allows rapid and flexible responses to events at the cell surface (e.g. signal platelets to initiate an interaction with coagulation factors).

Several types of integrins exist, and one cell generally has multiple different types on its surface. Integrins are found in all animals while integrin-like receptors are found in plant cells.

Integrins work alongside other proteins such as cadherins, the immunoglobulin superfamily cell adhesion molecules, selectins and syndecans, to mediate cell-cell and cell-matrix interaction. Ligands for integrins include fibronectin, vitronectin, collagen and laminin. Specific integrins contemplated by the inventors include α₄β₁ integrin, α₂β₁ integrin, α₃β₁ integrin, α₇β₁ integrin, and/or α₉β₁ integrin.

The inventors first characterized the native expression of integrin across a library of cell types to identify what is endogenously present and absent. Based on those profiles, integrin subunit expression was either knocked in or knocked out using CRISPR-Cas9 and integrin subunit-targeted guide RNAs. For knock-in cells, integrin coding DNA was inserted into the AAVS1 safe harbor locus. For knock-out cells, base pair insertions or deletions at the beginning of the integrin coding gene rendered integrin proteins nonfunctional. Cells were single cell sorted, expanded, and sequenced to confirm cell editing.

In another embodiment, the cells could instead be modified by introducing gene expression regulatory elements. For example, one can introduce a coding region for an exogenous integrin, i.e., one the cell does not normally produce, under the control of an inducible promoter.

### II. Cell Engineering/Editing

In some embodiments, cells are used without engineering if they naturally express or do not express the appropriate adhesive protein. Alternatively, the inventors engineer cells to create specific patterns of expression of adhesive proteins while creating scaffolds that present complementary component to those adhesive proteins found on the cells (e.g., integrin-binding peptides), thereby permitting precise control of cell placement since cells that lack the requisite protein for binding will be unable bind and/or to proliferate. The implementation of this strategy involves the increase or decrease of adhesive protein expression which can be done permanently through genetic engineering or transiently through expression modification.

In one aspect, alpha and beta integrins form heterodimers, which enable cells to bind to the extracellular matrix (ECM) and other proteins. These integrins bind to extracellular matrix proteins at specific binding pockets. The inventors can mimic these pockets of adhesion with peptide sequences (or conceivably full proteins) that integrins will still bind. These peptides can bind to one integrin heterodimer or many. They aim to use peptide sequences that uniquely bind to one integrin heterodimer. However, cells endogenously express many integrins and have heavily overlapping integrin expression profiles, preventing the potential for unique integrin-peptide bonding. Therefore, they will simultaneously engineer cells to have a specific integrin expression profile that enable only a single cell type used in the tissue to adhere to the integrin-binding peptide sequences patterned on the scaffold to enable that cell type to uniquely adhere to that area. Conversely, that cell will lack (either endogenously and/or through engineering) the integrins necessary to adhere to other regions of the scaffolds patterned with other integrin-biding peptides and therefore remain in designated areas.

Cells can be engineered/edited using a variety of CRISPR systems. Cas (CRISPR associated protein) molecules play a vital role in the immunological defense of certain bacteria against DNA viruses and plasmids and are heavily utilized in genetic engineering applications. Its main function is to cut DNA and thereby alter a cell's genome.

Cas9 is perhaps the most studied of all the Cas molecules. It is a dual RNA-guided DNA endonuclease enzyme associated with the Clustered Regularly Interspaced Short Palindromic Repeats (CRISPR) adaptive immune system in Streptococcus pyogenes. S. pyogenes utilizes CRISPR to memorize and Cas9 to later interrogate and cleave foreign DNA, such as invading bacteriophage DNA or plasmid DNA. Cas9 performs this interrogation by unwinding foreign DNA and checking for sites complementary to the 20 bP spacer region of the guide RNA. If the DNA substrate is complementary to the guide RNA, Cas9 cleaves the invading DNA. In this sense, the CRISPR-Cas9 mechanism has a number of parallels with the RNA interference (RNAi) mechanism in eukaryotes.

Apart from its original function in bacterial immunity, the Cas9 protein has been heavily utilized as a genome engineering tool to induce site-directed double-strand breaks in DNA. These breaks can lead to gene inactivation or the introduction of heterologous genes through non-homologous end joining and homologous recombination respectively in many laboratory model organisms. Alongside zinc finger nucleases and Transcription activator-like effector nuclease (TALEN) proteins, Cas9 is becoming a prominent tool in the field of genome editing.

Cas9 has gained traction in recent years because it can cleave nearly any sequence complementary to the guide RNA. Because the target specificity of Cas9 stems from the guide RNA:DNA complementarity and not modifications to the protein itself (like TALENs and zinc fingers), engineering Cas9 to target new DNA is straightforward. Versions of Cas9 that bind but do not cleave cognate DNA can be used to locate transcriptional activator or repressors to specific DNA sequences in order to control transcriptional activation and repression. Native Cas9 requires a guide RNA composed of two disparate RNAs that associate—the CRISPR RNA (crRNA), and the trans-activating crRNA (tracrRNA). Cas9 targeting has been simplified through the engineering of a chimeric single guide RNA (chiRNA).

Other useful Cas proteins include Cas6, AsdCas12a, SpdCas9, CjdCas9, and SadCas9.

Delivery systems for the CRISPR-based systems of the present disclosure include integrative vectors such as retroviral or lentiviral system, or transient delivery systems, such as adeno-associated virus (AAV) or non-integrating lentiviruses. In embodiments, the AAV vector is replication-defective or conditionally replication defective. In embodiments, the AAV vector is a recombinant AAV vector. In some embodiments, the AAV vector comprises a sequence isolated or derived from an AAV vector of serotype AAV1, AAV2, AAV3, AAV4, AAV5, AAV6, AAV7, AAV8, AAV9, AAV10, AAV11 or any combination thereof.

Delivery systems also include non-viral methods such as lipid nanoparticles and/or mRNA delivery, in particular well known lipofection and electroporation techniques can be employed.

Cells can also be edited with siRNA/mRNA to transiently knock in/knock out integrin expression, allowing cells to resume their endogenous protein expression after self-organizing. Other endogenous proteins (e.g., selectins, cadherins) can be knocked in or knocked out and likely function in a similar way. Engineered chimeric proteins can be inserted into cells so the extracellular portion binds specifically to a peptide/protein/molecule on the hydrogel while the intracellular portion is engineered to start a finely tuned cellular response.

Sorting of cells is typically performed by fluorescent cell sorting. When single cell colonies of interest are identified, sequencing using any suitable approach will confirm the underlying genetic changes.

In one embodiment, to create knock-out cell lines, cells with high expression of target integrins were determined using flow cytometry. Once positive integrin cell lines were identified, gRNAs were designed and ordered. The most efficient gRNA was elucidated through a cell based cutting assay. The chosen gRNA was then transfected into the positive integrin cell line with Cas9 using lipofection or electroporation. Cells were sorted after 48-72 hours using fluorescence-activated cell sorting (FACS). Cells were incubated with the corresponding anti-integrin antibody and cells showing no detectable shift in antibody signal were single-cell sorted into 96 well plates. Cells were then allowed to expand to create clonal populations for 3-4 weeks. Clonal cell colonies were screened using flow cytometry and only those colonies that show no shift in antibody signal were expanded further. PCR was performed on those colonies to extract the target edited region of the knocked-out integrin. The PCR amplicons were Sanger sequenced to confirm gene editing, by way of base pair insertions or deletions.

To create knock-in cell lines, cells with no discernable expression of target integrins were determined using flow cytometry. The DNA coding for the desired knock-in integrin was inserted into the AAVS1 safe harbor locus. An antibiotic resistance gene was inserted with the donor DNA for antibiotic selection of stably transfected cells. Another option is adding a fluorescent reporter protein to label stably transfected cells. The most efficient gRNA, Cas9 and the integrin coding DNA will be transfected into the cell line using lipofection or electroporation. Cells were allowed to recover for 72 hours before being exposed to the chosen antibiotic. Cells were expanded and flow cytometry was used to confirm integrin expression. Cells were bulk sorted for the highest integrin expressing population using FACS and expanded for 2-3 weeks. PCR was performed on those colonies to extract the target edited region of the AAVS1 safe harbor locus. The PCR amplicons were sanger sequenced to confirm gene editing, showing insertion of a new DNA sequence.

### III. Scaffolds

Tissue scaffold can be made of any natural or synthetic polymer, as long as peptides can be conjugated to the polymers. There are other ways to attach peptides as well. There are also small molecules known to bind to integrins (as well as other proteins), so it does not necessarily have to be peptides or proteins on the scaffold. The scaffold can have degradable linkers for cellular remodeling. Importantly, the scaffolds do not possess sequences/features that would lead to undesired cell binding, undermining peptide-mediated control.

A polymer may be a naturally occurring polymer or a synthetic polymer. For example, a polymer may comprise polystyrene, polyester, polycarbonate, polyethylene, polypropylene, polyfluorocarbon, nylon, polyacetylene, polyvinyl chloride (PVC), polyolefin, polyurethane, polyacrylate, polymethacrylate, polyacrylamide, polymethacrylamide, polymethyl methacrylate, poly(2-hydroxyethyl methacrylate), polysiloxane, polydimethylsiloxane (PDMS), polyhydroxyalkanoate, PEEK®, polytetrafluoroethylene, polyethylene glycol, polysulfone, polyacrylonitrile, collagen, cellulose, cellulosic polymers, polysaccharides, polyglycolic acid, poly(L-lactic acid) (PLLA), poly(lactic glycolic acid) (PLGA), polydioxanone (PDA), poly(lactic acid), hyaluronic acid, agarose, alginate, chitosan, or a blend or copolymer thereof. In an embodiment, the implantable construct comprises a polysaccharide (e.g., alginate, cellulose, hyaluronic acid, or chitosan). In an embodiment, the encapsulated cell composition comprises alginate.

Hyaluronan (HA) is a glycosaminoglycan present in many tissues throughout the body that plays an important role in embryonic development, wound healing, and angiogenesis. In addition, HA interacts with cells through cell-surface receptors to influence intracellular signaling pathways. Together, these qualities make HA attractive for tissue engineering scaffolds. HA can be modified with crosslinkable moieties, such as methacrylates and thiols, for cell encapsulation. Crosslinked HA gels remain susceptible to degradation by hyaluronidase, which breaks HA into oligosaccharide fragments of varying molecular weights. Auricular chondrocytes can be encapsulated in photopolymerized HA hydrogels where the gel structure is controlled by the macromer concentration and macromer molecular weight. In addition, photopolymerized HA and dextran hydrogels maintain long-term culture of undifferentiated human embryonic stem cells. HA hydrogels have also been fabricated through Michael-type addition reaction mechanisms where either acrylated HA is reacted with PEG-tetrathiol, or thiol-modified HA is reacted with PEG diacrylatec.

Chondroitin sulfate makes up a large percentage of structural proteoglycans found in many tissues, including skin, cartilage, tendons, and heart valves, making it an attractive biopolymer for a range of tissue engineering applications. Photocrosslinked chondroitin sulfate hydrogels can be prepared by modifying chondroitin sulfate with methacrylate groups. The hydrogel properties were readily controlled by the degree of methacrylate substitution and macromer concentration in solution prior to polymerization. Further, the negatively charged polymer creates increased swelling pressures allowing the gel to imbibe more water without sacrificing its mechanical properties. Copolymer hydrogels of chondroitin sulfate and an inert polymer, such as PEG or PVA, may also be used.

Polyethylene glycol (PEG) has been the most widely used synthetic polymer to create macromers for cell encapsulation. A number of studies have used poly(ethylene glycol) di(meth)acrylate to encapsulate a variety of cells. Biodegradable PEG hydrogels can be prepared from triblock copolymers of poly(α-hydroxy esters)-b-poly(ethylene glycol)-b-poly(α-hydroxy esters) endcapped with (meth)acrylate functional groups to enable crosslinking. PLA and poly(8-caprolactone) (PCL) have been the most commonly used poly(α-hydroxy esters) in creating biodegradable PEG macromers for cell encapsulation. The degradation profile and rate are controlled through the length of the degradable block and the chemistry. The ester bonds may also degrade by esterases present in serum, which accelerates degradation.

Biodegradable PEG hydrogels can also be fabricated from precursors of PEG-bis-[2-acryloyloxy propanoate]. As an alternative to linear PEG macromers, multi-arm PEG-polymers of poly(glycerol-succinic acid)-PEG, which contain multiple reactive vinyl groups per PEG molecule, can be used. An attractive feature of these materials is the ability to control the degree of branching, which consequently affects the overall structural properties of the hydrogel and its degradation. Degradation will occur through the ester linkages present in the dendrimer backbone.

The biocompatible, hydrogel-forming polymer can contain polyphosphoesters or polyphosphates where the phosphoester linkage is susceptible to hydrolytic degradation resulting in the release of phosphate. For example, a phosphoester can be incorporated into the backbone of a crosslinkable PEG macromer, poly(ethylene glycol)-di-[ethylphosphatidyl (ethylene glycol) methacrylate] (PhosPEG-dMA), to form a biodegradable hydrogel. The addition of alkaline phosphatase, an ECM component synthesized by bone cells, enhances degradation. The degradation product, phosphoric acid, reacts with calcium ions in the medium to produce insoluble calcium phosphate inducing autocalcification within the hydrogel. Poly(6-aminoethyl propylene phosphate), a polyphosphoester, can be modified with methacrylates to create multivinyl macromers where the degradation rate was controlled by the degree of derivatization of the polyphosphoester polymer.

In one embodiment, integrin-specific cell adhesion peptides were synthesized with a cysteine added to the N-terminus with a glycine spacer using solid-phase peptide synthesis, and purified using high performance liquid chromatography. The N-terminal cysteine allows for conjugation of the peptide to the polyethylene glycol diacrylate (PEG-DA) hydrogel scaffold through a thiol-ene reaction. Peptides are patterned/conjugated to the hydrogel in any design through UV crosslinking with a photoinitiator. This can be done in two separate steps, the first of which adds the peptide to some PEG-DA molecules and the second of which provides a photo-reaction in which the peptide conjugated PEG-DA reacts with other PEG-DA molecules to form a gel. Gel formation can also be done in one step, where the peptide and PEG-DA molecules react simultaneously with each other.

### IV. Methods and Applications

This process is designed to spatially direct the adhesion and proliferation of multiple cell types, within a complex tissue. The scaffold can be patterned to precisely place multiple interacting cell types in a tissue or organ. It should be noted that integrin binding is the easiest version of this approach, but it could be extended to other cell binding motifs (e.g., selectins, cadherins) or using de novo or chimeric proteins that enable adhesion.

The present inventors envision the methods and compositions described herein to create self-organizing tissues/organs that better recapitulate native tissue structure and function. The goal is to create tissues/organs that could not only be used for transplantation, but also used for more accurate in vitro experiments (e.g., drug screening, cancer models) given the ability to better mimic the complexity of tissues and organs and to function more like native tissue. Other uses include producing food products synthetically.

### V. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

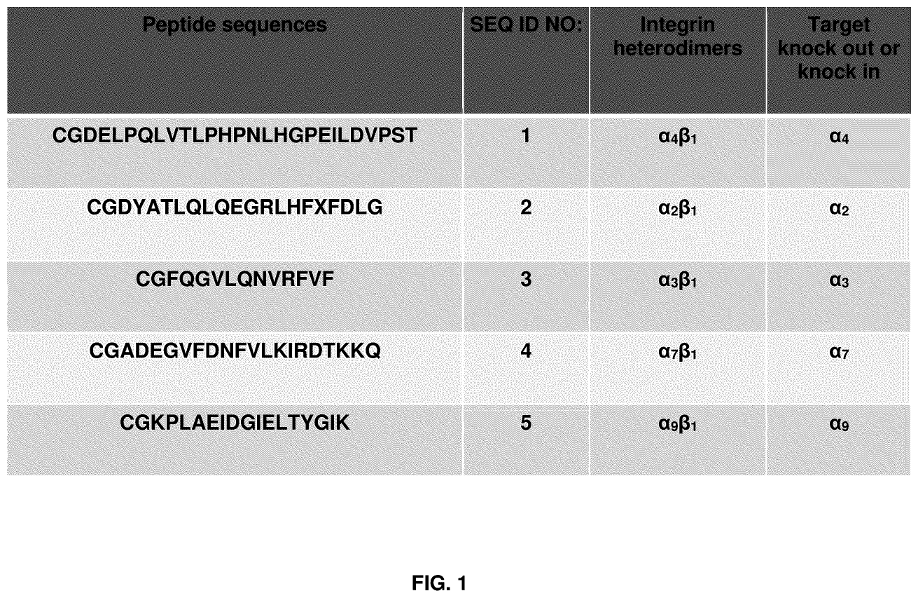

Integrin-specific cell adhesion peptides were identified in the literature (FIG. 1), synthesized with a cysteine added to the N-terminus using solid-phase peptide synthesis, and purified using high performance liquid chromatography. To determine native integrin expression, flow cytometry was performed on several human cell types using several integrin heterodimer-specific antibodies. To confirm unique cell-peptide adhesion, the inventors performed plate-based cell adhesion assays. Peptides were conjugated to IgG using a crosslinker for presentation at the bottom of the well. Cells expressing or lacking certain integrins were seeded in wells coated with peptide and cell spreading and adhesion was quantified. Integrin expression was either knocked in or knocked out using CRISPR-Cas9 and integrin subunit-targeted guide RNAs. For knock-in cells, integrin coding DNA was inserted into the AAVS1 safe harbor locus. For knock-out cells, base pair insertions or deletions at the beginning of the integrin coding gene rendered integrin proteins nonfunctional. Cells were sorted, expanded, and sequenced to confirm cell editing. Cells confirmed to be edited were then used for adhesion assays on peptides.

### Example 2—Results

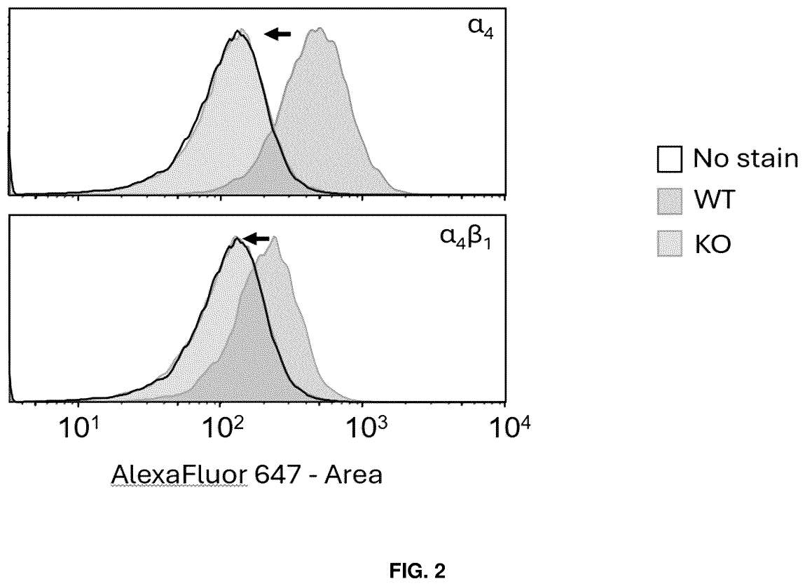

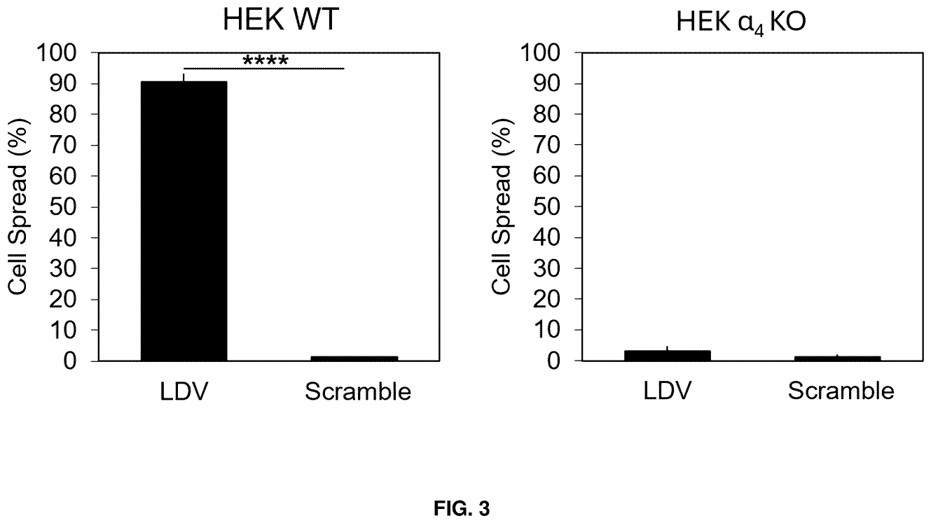

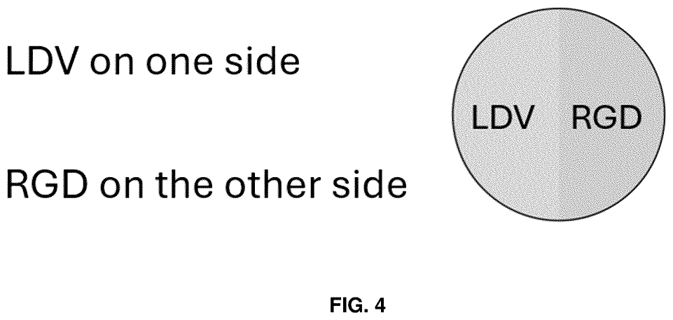

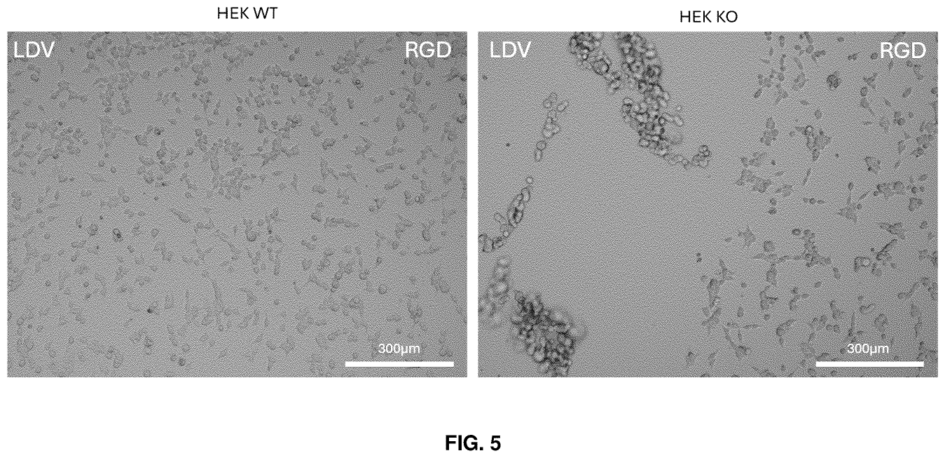

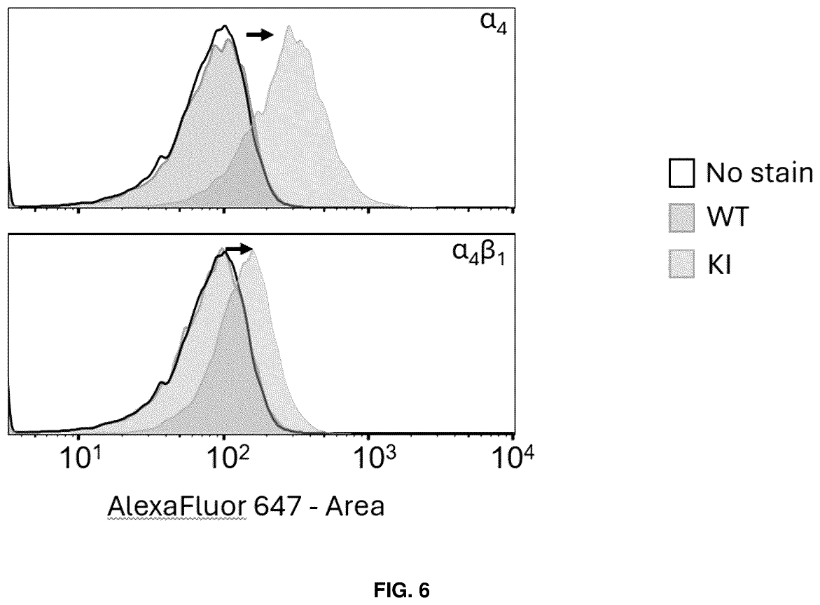

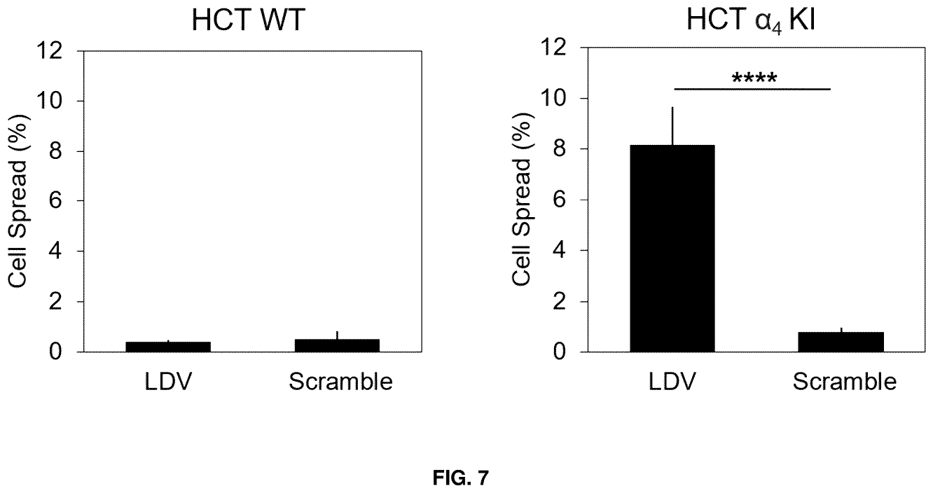

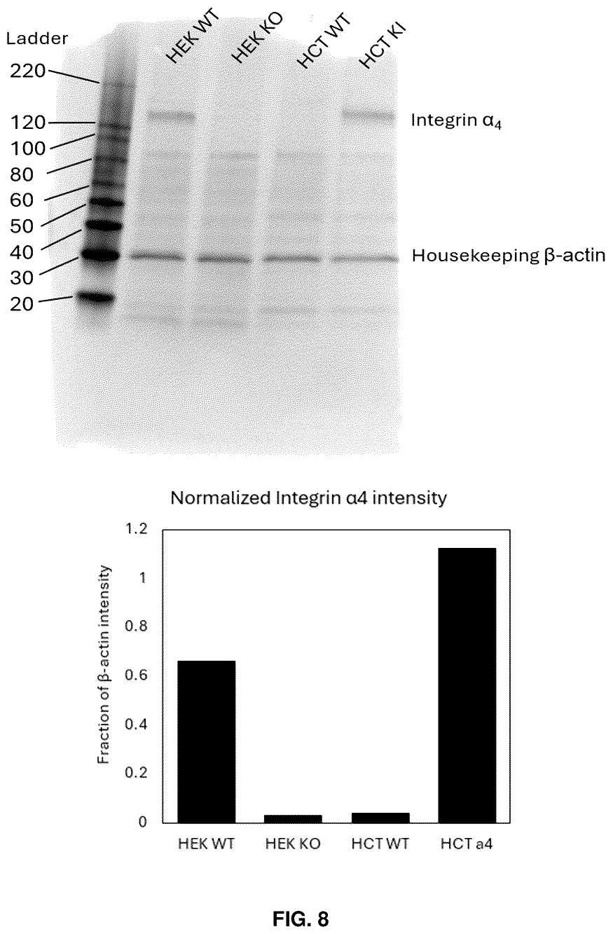

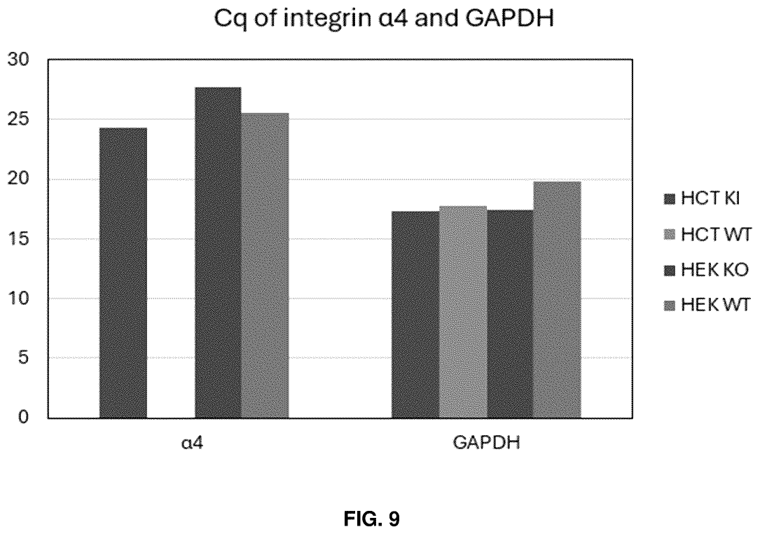

Integrin α₄ was successfully knocked out of HEK293 cells that endogenously express the α₄β₁ heterodimer to prevent peptide binding through α₄β₁. Integrin α₄ was chosen instead of β₁ to minimize potentially undesirable side effects since the latter is part of 12 heterodimers instead of only 2 for α₄. Flow cytometry confirmed the difference in α₄β₁ expression between parent cells and otherwise-equivalent α₄ knock-out cells (FIG. 2). Sequencing results confirmed the insertion of a single base pair at the Cas9 cut site, resulting in a frameshift for the rest of the integrin 4 coding sequence. In the plate-based adhesion assay, wild-type HEK 293 cells were able to bind and spread on LDV and α₄β₁ integrin-specific peptide, while knock out HEK 293 cells unable to (FIG. 3). HEK cells were unable to bind to a scramble peptide regardless of α₄ expression status (FIG. 3). HCT-116 cells, which do not express α₄β₁, were unable bind to LDV or the scramble peptide (FIGS. 6-7). In contrast, cells that do not inherently express α₄, but had it knocked-in, gained the ability to bind to the LDV peptide (FIGS. 6-7). When seeded onto a surface, α₄⁺ cells bind to and spread on the LDV-presenting pattern but are unable to bind to regions patterned with the scramble peptide (FIG. 3, FIG. 7). These results show that the inventors are able to pattern a surface with a unique cell adhesion peptide and change a cell's ability to bind to the surface by knocking in or knocking out a relevant integrin subunit. The inventors show that when a surface is patterned with the widely recognized RGD peptide, cells are able to bind to the RGD portion. However, if half of surface is patterned with LDV, then only α₄⁺ cells are able to bind to the entire surface, whereas α₄⁻ cells are only able to bind to the RGD half (FIGS. 4-5). Cell integrin expression was also confirmed using western blot (FIG. 8) and qPCR (FIG. 9). After establishing proof of concept with α₄, the inventors have shown successful integrin-specific cell adhesion to peptides for α₂β₁ and are currently generating α₂-knockout cells to show loss of binding. In summary, the inventors have developed a method to harness the power of unique integrin-peptide interactions, which may enable cells to self-organize and maintain their localization within scaffolds engineered for complex tissues due to integrin-mediated restriction of adhesion.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A composition comprising a scaffold material patterned with one or more target molecules that bind(s) a cell adhesion protein.

2. The composition of claim 1, wherein the cell adhesion protein is an integrin, a selectin or a cadherin.

3. The composition of claim 2, wherein the integrin is α₄β₁ integrin, α₂β₁ integrin, α₃β₁ integrin, α₇ β₁ integrin, and/or α₉β₁ integrin.

4. The composition of claim 3, wherein the target molecule(s) is/are a fibronectin peptide, such as LDV.

5. The method of claim 1, wherein the target molecule(s) is/are attached to the scaffold by direct attachment or through a linker, such as a degradable linker.

6. The composition of claim 1, wherein the scaffold material is polymer, such as hydrogel, such an alginate hydrogel.

7. The composition of claim 1, wherein a plurality of cells is patterned on said scaffold through binding to said target molecule, wherein said plurality of cells have an identical genotype/phenotype.

8. The composition of claim 1, wherein a plurality of cells is patterned on said scaffold through binding to said target molecule, wherein said plurality of cells have more than one genotype/phenotype.

9. The composition of claim 7, wherein the plurality of cells is engineered to not express an integrin, such as α₄β₁ integrin, α₂β₁ integrin, α₃β₁ integrin, α₇β₁ integrin, and/or α₉β₁ integrin, a selectin or a cadherin, such as E-cadherin or P-selectin.

10. The composition of claim 7, wherein the plurality of cells is engineered to express an integrin, such as α₄β₁ integrin, α₂β₁ integrin, α₃β₁ integrin, α₇β₁ integrin, and/or α₉β₁ integrin, a selectin and a cadherin and to lack expression of an integrin, a selectin or a cadherin.

11. A method of preparing a composition according to claim 1, comprising:
(a) providing a scaffold; and
(b) patterning one or more targets molecules that bind a cell adhesion protein on a surface of the scaffold.

12. The method of claim 11, further comprising contacting the patterned scaffold with a plurality of cells, wherein (a) said plurality of cells have an identical genotype/phenotype, or (b) wherein said plurality of cells more than one genotype/phenotype.

13. The method of claim 12, wherein the plurality of cells is engineered to express and/or not express one or more cell adhesion peptides (an integrin, a selectin, a cadherin, e.g., E-cadherin or P-selectin), proteins, or small molecules present on the scaffold.

14. The method of claim 12, wherein the plurality of cells is engineered to express one or more cell adhesion peptides (an integrin, a selectin, a cadherin, e.g., E-cadherin or P-selectin), proteins, or small molecules present on the scaffold.

15. The method of claim 12, wherein the plurality of cells is engineered to not express one or more cell adhesion peptides (an integrin, a selectin, a cadherin, e.g., E-cadherin or P-selectin), proteins, or small molecules present on the scaffold.

16. The method of claim 13, wherein the integrin integrin is α₂β₁ integrin, α₃β₁ integrin, α₇β₁ integrin, α₇β₁ integrin, and/or α₉β₁ integrin.

17. An artificial tissue or organ produced by culturing the composition of claim 7.


SEQUENCE LISTING (5 sequences)

SEQ ID NO: 1 - protein, 27 aa, synthetic construct
CGDELPQLVTLPHPNLHGPEILDVPST

SEQ ID NO: 2 - protein, 21 aa, synthetic construct
CGDYATLQLQEGRLHFXFDLG

SEQ ID NO: 3 - protein, 14 aa, synthetic construct
CGFQGVLQNVRFVF

SEQ ID NO: 4 - protein, 21 aa, synthetic construct
CGADEGVFDNFVLKIRDTKKQ

SEQ ID NO: 5 - protein, 18 aa, synthetic construct
CGKPLAEIDGIELTYGIK